(19) United States
(12) Patent Application Publication
(10) Pub. No.: US 2006/0205015 A1
(43) Pub. Date: Sep. 14, 2006

(54) Method for detecting the interactions between a G protein-coupled receptor (GPCR) and one of the Galpha or Gbetagamma subunits

(76) Inventors: Herve Ansanay, Tavel (FR); Michel Fink, Bagnols Sur Ceze (FR); Gerard Mathis, Bagnols Sur Ceze (FR); Damien Maurel, Marguerittes (FR); Eric Trinquet, Pont Saint Esprit (FR); Jean-Philippe Pin, Montpellier (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/352,398

(22) Filed: Feb. 13, 2006

Related U.S. Application Data
(60) Provisional application No. 60/651,987, filed on Feb. 14, 2005.

(51) Int. Cl. G01N 33/53; C07F 5/00; C07K 14/705; C12N 5/06

(52) U.S. Cl. 435/7.1; 530/400; 534/15; 435/325

(57) ABSTRACT

The present invention relates to a method for the detection of ligands (agonists or antagonists) specific for a G protein-coupled receptor (GPCR), which comprises the steps consisting in: 1) bringing a receptor labelled with a member of a donor/acceptor pair into contact with a Gα or Gβγ subunit of a G protein labelled with the other member of the donor/acceptor pair; 2) measuring the transfer by proximity effect between the donor and the acceptor.

This application claims the benefit of the filing date of U.S. Provisional Application Ser. No. 60/651,987 filed Feb. 14, 2005 which is incorporated by reference herein.

### FIELD OF THE INVENTION

The present invention relates to a method for detecting the interactions between a G protein-coupled receptor (GPCR) and one of the Gα or Gβ subunits of a G protein by transfer by proximity effect between the two members of a donor/acceptor pair and its applications, such as in particular the screening for novel medicaments and the identification of orphan receptors.

In the present description, the expression “transfer by proximity effect” denotes a transfer of energy characterized by an FRET signal (HTRF® technology, CIS BIO INTERNATIONAL), a transfer of singlet oxygen (Alphascreen® technology, PerkinElmer, see for example Beaudet et al., Genome Res., 2001 Apr. 11 (4), 600-8), an electron transfer (SPA technology, Amersham Biosciences, see for example Udenfriend et al., Anal. Biochem., 1987, Mar., 161(2), 494-500).

In the techniques for fluorescence non-radiative energy transfer which are commonly called FRET techniques, the TR-FRET technique (Time-Resolved Fluorescence resonance energy transfer) allows measurement of time-resolved fluorescence in a homogeneous medium. The use of this technique with rare earth chelates or cryptates, developed in particular by G. Mathis et al. (see in particular “Homogeneous time resolved fluorescence energy transfer using rare earth cryptates as a tool for probing molecular interactions in biology”, Spectrochimica Acta Part A 57 (2001) 2197-2211) has numerous advantages which have already allowed several applications in the field of diagnostics in vitro and in that of screening at high throughput in the pharmaceutical industry.

This technique, also known by the name of HTRF® (Homogenous Time Resolved Fluorescence), uses a first donor fluorescent compound and a second acceptor fluorescent compound.

After light excitation at the wavelength of the donor, an energy transfer takes place between the donor and the acceptor if they are close to each other; this energy transfer is characterized by an emission of light by the acceptor (FRET signal) which may be measured with the aid of a fluorimeter.

The receptors, in particular the receptors with seven transmembrane domains coupled to G proteins (called hereinafter GPCR) are involved in numerous pathological processes. GPCRs are transmembrane proteins responsible for the recognition and transfer of information from the outside to the inside of the cell. These proteins represent major therapeutic targets and more than 50% of the current medicaments target these receptors or their transduction cascade.

The mechanisms for activation of the GPCRs in the presence of a ligand are schematically represented in the appended FIG. 1.

The binding of an agonist ligand to a GPCR modifies its tertiary structure, which, in turn, activates the G protein coupled to the GPCR. The activation of the G protein causes, depending on the case, the activation or inhibition of an effector which produces a second intracellular messenger such as cAMP or IP₃.

In the present description, the expression “orphan receptors” denotes transmembrane receptors whose DNA sequence suggests that they correspond to GPCR but whose specific natural ligand is not known.

The characterization of the signalling pathways (second messengers and effectors) and the identification of ligands for GPCRs termed “orphan receptors” is of great importance in understanding the physiological role of these GPCRs and for the production of new medicaments.

State of the Art

Various methods exist for the detection of the receptor/G protein interactions which use one of the components of the process for the activation of the GPCRs, in particular for the detection of specific ligands.

For example, one method for detecting ligands uses a non-hydrolysable radioactive derivative of GTP, namely [³⁵S]GTPγ which binds to the Gα protein when GPCR is activated. The kinetic parameters for the binding of the GTP analogue depend on the type of G protein. This radioactive method is not suitable for the screening of medicaments at high throughput.

Other conventional methods are based on the activation of the effectors by subunits of the activated trimeric G proteins.

As indicated in FIG. 1, the activation of the effectors and the production of second messengers such as cAMP or IP₃ are produced after the stimulation of GPCR and the G protein. This method therefore relates to substances produced downstream of the process for the activation of GPCR.

Methods, suitable for the screening of medicaments at high throughputs, use recombinant systems based on the measurement of an enzyme activity, such as the activity of β-galactosidase or of luciferase, whose expression is controlled by the second messengers produced by the activation of GPCR. These are indirect methods for measuring the activity of GPCR, which also relates to the substances produced downstream of the GPCR activation process.

Another method for the detection of ligands focuses on the translocation of arrestin-GFP.

Arrestin is involved in the regulatory machinery which shuts off the activation pathway triggered by the binding of the ligand to GPCR. It makes it possible to trigger the process for internalization of the receptor in the cell, which step is necessary for its degradation or its recycling on the cell membrane. The technology developed by NORAK BIOSCI (Transfluor) uses arrestin fused with a fluorescent protein (GFP) (arrestin-GFP) which is expressed in cells. Under normal conditions, arrestin-GFP is distributed in the cystosol. After activation of GPCR, arrestin-GFP will interact with the GPCR present at the membrane and stimulated by an agonist. Its cellular distribution will therefore be modified. It will leave the cytosol and become redistributed at the plasma membrane (phenomenon of translocation) and participate in the internalization phenomenon (disappearance of the receptors from the cell surface). Over time, the fluorescence of arrestin-GFP attached to its receptor can also be measured near the internal vesicles (endosomes) at the cell which indicate the phenomenon of endocytosis of certain receptors. These variations in the distribution of arrestin-GFP are achieved by virtue of microscopy and image processing techniques. For these reasons, this technique is not suitable for high throughput screening (HTS).

The recent work by Janetopoulos et al. [Science (2001) 291: 2408-2411; Methods (2002) 27: 366-373] has made it possible to visualize an FRET signal between the Gα₂-CFP and Gβ-YFP subunits in D. discoideum. The work by Azpiazu and Gautam [JBC (2004) 279 (26): 27709-27718] show similar results of variation in FRET signal between Gα-CFP and Gβ-YFP in the model for stimulation of the muscarinic receptors M2 and M3 expressed in the CHO cells. The Berlot team [JBC (2004) 279 (29): 30279-30286] has demonstrated, in vivo and by microscopy, the interactions between Gβ and Gγ, labelled with fragments of a fluorescent protein (YFP). The Gβγ combination restores the fluorescence of the YFP protein.

These observations suggest that the activation of the heterotrimeric G proteins may be analysed by the FRET technique. These techniques indirectly show the activation of a receptor by the measurement of FRET between the subunits of the G proteins but in no way show, and directly, the state of activation of the GPCR measured by a specific FRET signal between itself, the receptor of interest and either of the subunits of the G proteins to which it is coupled.

Another method consists in inserting, by molecular biology, fluorescent proteins (CFP and YFP) into a GPCR, at the level of the 3rd intracellular loop and of the C-terminal part respectively, and in measuring, by the variation of an FRET signal, the activation of the receptor. This method is described by Vilardaga et al. [Nat. Biotechnol. (2003) 21: 807-812; WO 2004 057333 A1]. It does not directly take into account the GPCR-G protein interactions and the modulations of these interactions following the activation of GPCR.

Another method consists in detecting the oligomerization of the GPCRs by the FRET technique (patent U.S. Pat. No. 6,824,990 B1).

None of these methods makes it possible to account, by measuring an FRET signal, for the state of premature activation of a GPCR receptor, termed receptor of interest, and for either of the subunits of the G proteins to which it is coupled.

### SUMMARY OF THE INVENTION

It has now been found, surprisingly, that it is possible to detect the activation of a G protein-coupled receptor (GPCR) at an early stage of the activation process by measuring the fluorescence signal based on a transfer of proximity effect between a receptor and one of the Gα or Gβγ subunits of a G protein.

The measurement of the signal for the transfer of proximity effect is based on the state of activation of a given GPCR/Gα pair or of a given RCPG/Gβγ pair. This signal is therefore highly specific since it is generated by a unique pair necessarily composed of a GPCR of interest and of one of the Gα or Gβγ subunits of the corresponding G protein. This signal for the transfer of proximity effect may be modulated, positively or negatively, when the receptor is specifically stimulated by a pharmacological agent (agonist, inverse agonist, antagonist or allosteric regulator).

The invention also offers an advantage from the point of view of the specificity of the response since it considers an early event in the pathway for the activation of the GPCR of interest. Unlike the methods already known, it is positioned considerably upstream of the signalling pathway for the GPCRs and takes into account close interactions between GPCR/G protein, in the absence or in the presence of potential pharmacological agents.

Thus, the subject of the present invention is a method for the detection of the interactions between a receptor and one of the Gα or Gβγ subunits of a G protein, which comprises the steps consisting in:

1) bringing a receptor labelled with a member of a donor/acceptor pair into contact, in a biological medium, with a Gα or Gβγ subunit of a G protein labelled with the other member of the donor/acceptor pair;

2) measuring the transfer by proximity effect between the donor and the acceptor.

The method of the invention, which makes it possible to detect the interactions between a receptor and one of the Gα or Gβγ subunits of a G protein, is particularly appropriate for:

1) demonstrating the receptor/G protein coupling,

2) the study of the properties of the receptors;

3) the detection of agonist or antagonist ligands for the receptors and the screening of novel medicaments;

4) the identification of orphan receptors.

The bringing of the labelled receptor into contact with one of the Gα or Gβγ subunits of a G protein may be carried out in various ways, for example by coexpressing in cells nucleic acids encoding the receptor, on the one hand, and one of the Gα and Gβγ subunits, on the other hand, or by reconstituting a functional GPCR (comprising the subunits forming GPCR and the subunits of the G proteins) obtained by heterologous expression of nucleic acids encoding the said subunits forming the receptor, on the one hand, and the subunits encoding the subunits of the G proteins, on the other hand, or after purification from tissue extracts (see for example Florio et al. JBC 1985, Vol. 260 No. 8, pp. 3477-3483; Haga K. et al. JBC 1986, Vol. 261 No. 22, pp. 10103-10140; Devesa F. et al. 2002 Eur. J. Biochem, 269, pp 5163-5174; Stenlund et al. Anal. Biochem. 2003, 316, 243-250 and Park PSH.2004 FEBS Letters, 567, 344-348) in artificial plasma membranes.

Advantageously, when the GPCR used is a known receptor, the bringing into contact is carried out in a first instance in the presence of a natural ligand for the said GPCR which triggers the activation of the said receptor, and then in the presence of a potential pharmacological agent or any other test molecule capable of modulating the activity of the said receptor.

When the GPCR is an orphan receptor, the bringing into contact is carried out in the presence of a natural ligand for a known receptor which will make it possible, if there is interaction, to identify the said orphan receptor.

The detection of a potential pharmacological agent or any other test molecule, capable of modulating the activity of the said orphan receptor, is carried out as indicated above for a known receptor.

The members of the donor/acceptor pair used in the method of the invention are chosen according to the mode of transfer by proximity effect which it is desired to measure.

Thus, if the transfer by proximity effect is a transfer of energy by fluorescence, the donor/acceptor pair will consist of an energy-donating fluorophore and an energy-accepting fluorophore.

The appropriate fluorophores for the purposes of the invention are fluorescent molecules, chosen from fluorescent proteins or organic fluorophores. Examples of these fluorescent molecules are rhodamines, cyanines, squaraines, the fluorophores known by the name BODIPYs, such as for example difluoroboradiazaindacene derivatives, fluoresceins, the compounds known by the name AlexaFluor, rare-earth metal chelates, rare-earth metal cryptates, nanocrystals of the Quantum dot type, fluorescent proteins such as the green fluorescent protein (GFP) or its variants, fluorescent proteins extracted from corals, phycobiliproteins, such as B-phycoerythrin, R-phycoerythrin, C-phycocyanin, allophycocyanins, in particular those known by the name XL665 or the fluorescent compounds described in WO 2003/104685. Persons skilled in the art are capable of choosing the appropriate photocompatible donor/acceptor pairs for measurements of FRET or TR-FRET.

In the case of a transfer of singlet oxygen, the reagents associated with the Alphascreen® technology marketed by the company Perkin Elmer will be used for example as donor/acceptor pair (see Beaudet et al., Genome Res., 2001 Apr. 11 (4), 600-8).

If the transfer by proximity effect is a transfer of electrons, those described in Anal. Biochem., 1987, Mar., 161(2), 494-500 will be used as donor/acceptor pair.

The receptor used in the method of the invention is advantageously a known GPCR, such as the muscarinic receptor, the vasopressin receptor, the GABA (gamma-aminobutyric acid) receptor or a GPCR whose function it is desired to characterize, namely an orphan receptor.

The DNA sequences encoding these receptors may be amplified by PCR according to conventional molecular biology techniques known to persons skilled in the art (see Sambrook et al. below) or are commercially available (Invitrogen).

The Gα and Gβγ subunits to be used in the method of the invention may be the Gi, Gs, Gq or G12/G13, G14, G15 or G16 subunits or chimeric G proteins (with a Gqx type structure (it being possible for x to be mainly s, i, o) (see Conklin et al. Nature, 1993, v363 pp274-6; Milligan and Rees Trends Pharmacol Sci., 1999, v20 pp 118-24) whose sequences and properties are known to persons skilled in the art.

The DNA sequences encoding subunits may be amplified by PCR according to conventional molecular biology techniques known to persons skilled in the art (see Sambrook et al. below) or are commercially available (Invitrogen).

The labelling of the receptor and of one of the Gα or Gβγ subunits is carried out either directly by one or more covalent bonds according to techniques well known to persons skilled in the art (Janetopoulos et al., Methods (2002) 27: 366-373 and Vilardaga et al., Nat. Biotechnol. (2003) 21: 807-812 for example), or indirectly via binding partners of the tag/anti-tag antibody, antigen/antibody, avidin or streptavidin/biotin; haptene/antibody type.

The measurement of the signal for the transfer of proximity effect is carried out according to conventional methods well known to persons skilled in the art and described for example in the articles mentioned above.

For example, the variations of the FRET signal may be measured quantitatively by conventional fluorescence detectors commonly used by persons skilled in the art in laboratories specialized in high throughput screening (RUBYSTAR, BMG labtech). The measurement of the variations of the FRET signal makes it possible to directly account for the state of activation or inactivation of a GPCR and therefore presents a suitable solution for the search for molecules modulating the function of these receptors in a high throughput screening.

Advantageously, step 1) of the method of the invention is carried out by transfecting cells with a nucleic acid encoding a receptor and with a nucleic acid encoding a Gα or Gβγ subunit of a G protein, the receptor and the Gα or Gβγ subunit being coupled to a member of a donor/acceptor pair, for one, and to the second member of the said donor/acceptor pair, for the other.

According to one variant embodiment, step 1) of the method consists in bringing the preparations of cells transfected with a nucleic acid encoding a receptor and with a nucleic acid encoding a Gα or Gβγ subunit of a G protein, the receptor and the Gα or Gβγ subunit being coupled to a member of a donor/acceptor pair, for one, and to the second member of the said donor/acceptor pair, for the other, into contact with a test substance.

Advantageously, step 2) of the method of the invention is carried out as follows:

1) the transfer by a proximity effect between the donor and the acceptor is measured in the absence of ligand, of pharmacological agents for the said receptor and of test molecules (basal signal);

2) the signal obtained in the presence of a specific ligand for this GPCR which will trigger the activation of this receptor (reference signal) is optionally compared to the basal signal;

3) the signal obtained in the presence of pharmacological agents or of chemical molecules which are capable of modulating the activity of the said receptor is compared to the basal and reference signals.

According to a preferred variant embodiment of the method of the invention, the donor/acceptor pair is a pair consisting of a donor fluorophore and an acceptor fluorophore and the signal measured is the FRET signal emitted after light excitation of the measurement medium at the excitation wavelength of the donor fluorophore.

The subject of the present invention is also the cells transfected with a nucleic acid sequence encoding a GPCR and with a nucleic acid sequence encoding a Gα or Gβγ subunit of the corresponding G protein, the GPCR receptor and the Gα or Gβγ subunit being coupled to a donor fluorophore, for one, and to an acceptor fluorophore, for the other.

The subject of the invention is also the membrane preparations of transfected cells as defined above, which are obtained according to the conventional methods well known to persons skilled in the art.

Finally, the subject of the invention is a kit which comprises the preparations of transfected cells as defined above, and the means necessary for measuring the FRET signal.

### DETAILED DESCRIPTION OF THE INVENTION

The invention will now be described in greater detail with reference to the FRET technique without, as a result, limiting the present application to this single technique.

a) Preparations of Transfected Cells

In the present description, the expression “preparation of transfected cells” denotes the transfected cells themselves, the transfected cells permeabilized according to methods known to persons skilled in the art and illustrated in Example 1 and membrane preparations of transfected cells or membrane preparations obtained by reconstituting, the Gα subunits and the Gβγ subunits, and the receptors in artificial plasma membranes.

Cells may be stably transfected, that is to say that the sequences encoding the receptor or the Gα or Gβγ subunits are integrated into the genomic DNA of the cells.

Cells may also be transiently transfected with the aid of an expression vector of the plasmid type containing the nucleic acid sequence encoding the receptor and the nucleic acid sequence encoding one of the Gα or Gβγ subunits.

The techniques for transfecting cells, which are known to persons skilled in the art, are described for example in the manual by Sambrook et al., Molecular Cloning: A Laboratory Manual; 2^{nd} edition; Cold Spring Harbor Laboratory, Cold Spring Harbor, N.Y. (1989). The cells used are eukaryotic cells, such as for example HEK 293 or COS cells.

Transfection may also be carried out by heterologous expression in artificial plasma membranes.

Finally, the invention may be carried out on membrane preparations of transfected cells, whose preparation falls within the general knowledge of persons skilled in the art, and essentially comprises steps of breaking the cells using hypo-osmotic buffer, sonication, polytron, and recovering the membrane fractions of interest. These membrane preparations may be additionally enriched by additional steps of differential centrifugation or by gradient sedimentation.

b) Fluorophores

The donor or acceptor fluorophores (fluorescent compounds) which are suitable for the purposes of the invention are fluorescent substances chosen for example from rhodamines, cyanines, squaraines, fluorophores known by the name BODIPYs, fluoresceins, fluorophores known by the name AlexaFluor, rare-earth metal chelates, rare-earth metal cryptates, quantum dots, fluorescent proteins such as green fluorescent protein (GFP) or its variants, fluorescent proteins extracted from corals, phycobiliproteins, such as B-phycoerythrin, R-phycoerythrin, C-phycocyanin, allophycocyanins, in particular those known by the name XL665.

The donor fluorophores may be any of the above fluorophores which, when excited at a given wavelength, transfer energy to an acceptor fluorophore.

The acceptor fluorophores are any of the fluorophores, capable of emitting an FRET signal during the transfer of energy from the donor fluorophores.

The selection of the donor/acceptor fluorophore pair in order to obtain an FRET signal is within the capability of persons skilled in the art. Generally, for FRET to occur, the excitation spectrum of the acceptor has to at least partly cover the emission spectrum of the donor compound, and the transition dipoles of the donor and acceptor compounds must be parallel.

Donor-acceptor pairs which can be used to study the FRET phenomena are described in particular in the manual by Joseph R. Lakowicz (Principles of fluorescence spectroscopy, 2^{nd} edition 338), to which reference may be made by persons skilled in the art.

Preferably, the donor fluorophore is a rare-earth metal, in particular terbium or europium, complex.

Advantageously, the rare-earth metal complex is a chelate or a cryptate, preferably a cryptate with a pyridine unit.

Rare-earth metal chelates are described in particular in patents U.S. Pat. No. 4,761,481, U.S. Pat. No. 5,032,677, U.S. Pat. No. 5,055,578, U.S. Pat. No. 5,106,957, U.S. Pat. No. 5,116,989, U.S. Pat. No. 4,761,481, U.S. Pat. No. 4,801,722, U.S. Pat. No. 4,794,191, U.S. Pat. No. 4,637,988, U.S. Pat. No. 4,670,572, U.S. Pat. No. 4,837,169, U.S. Pat. No. 4,859,777. Other chelates are composed of a nonadentate ligand such as terpyridine (EP 403 593, U.S. Pat. No. 5,324,825, U.S. Pat. No. 5,202,423, U.S. Pat. No. 5,316,909).

The rare-earth metal cryptates are described in particular in patents EP 0 180 492, EP 0 601 113 and application WO 01/96 877.

Among these cryptates, most particularly preferred are the cryptates with a trisbipyridine unit, optionally substituted with carboxylate units, or the pyridine bisbipyridine cryptates of formula:

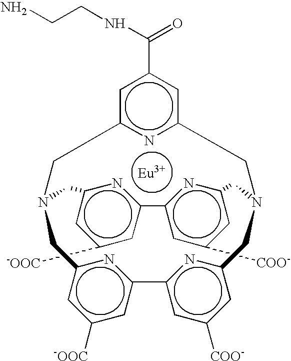

When the donor fluorophore is a rare-earth metal complex, the acceptor fluorophore is advantageously chosen from cyanines or allophycocyanine, optionally crosslinked.

c) Coupling of the Fluorophores

As indicated above, the fluorophores are coupled to the receptor or to the Gα or Gβγ subunits, either directly by one or more covalent bonds, or indirectly via bonding partners of the tag/anti-tag antibody, antigen/antibody, avidin or streptavidin/biotin; haptene/antibody type.

The direct coupling of the fluorophores may be carried out:

1) by expressing a fusion protein between the said receptor, the said Gα subunit, or the said Gβγ subunit and a fluorescent protein; or

2) by expressing a fusion protein between the said receptor, the said Gα subunit, or the said Gβγ subunit and a protein having an irreversible enzymatic activity (commonly called suicide enzyme) which transfers the fluorophore onto the GPCR, the Gα subunit, or the Gβγ subunit;

3) by splicing with an intein.

This technique of coupling by splicing with an intein is described for example in The Journal of Biological Chemistry, Vol. 273, No. 26, pp 15887-15890, 26 Jun. 1998 and J. Am. Chem. Soc. 2003, 125, 7180-7181.

The indirect coupling of the fluorophores may be carried out:

1) via a “ligand-receptor” or “tag/antitag” pair;

2) by expressing the “native” Gα subunit or Gβγ subunit receptors; in this case, the fluorescent compounds are conjugated with an antibody specifically recognizing the said receptor, the Gα subunit or the Gβγ subunit; or

3) by expressing the Gα subunit or Gβγ subunit receptors coupled to a fluorescent protein or to a suicide enzyme which transfers the fluorophore onto the said GPCR, the said Gα subunit or the Gβγ subunit;

4) by expressing the Gα subunit, or Gβγ subunit receptors bearing various TAGs; in this case, the fluorescent compounds are conjugated with antibodies directed specifically against the said TAGs.

These direct and indirect methods of coupling fluorophores use the recombinant DNA techniques well known to persons skilled in the art.

The direct coupling of a fluorophore by expressing a fusion protein between GPCR, the Gα subunit or the Gβγ subunit with a protein having an irreversible enzymatic activity advantageously uses, as protein with irreversible enzymatic activity, an O⁶-alkylguanine-DNA alkyl transferase (AGT) (see WO 02/083937) or a dehalogenase.

In this case, the cells are transfected with a nucleic acid encoding GPCR, with a nucleic acid encoding the Gα subunit or the Gβγ subunit and with a nucleic acid encoding the said protein with irreversible enzymatic activity. They are then exposed to the substrate specific for the said protein with enzymatic activity, which is covalently bound to a fluorophore which it is desired to couple to the receptor or to one of the Gα or Gβγ subunits.

In the indirect coupling of a fluorophore by means of a “ligand-receptor” or “tag/antitag” pair, a fusion protein is expressed between the receptor, the Gα subunit or the Gβγ subunit and a peptide sequence termed tag, the fluorescent compounds being in this case conjugated to an antibody specifically recognizing the tag. Use is made of the peptide sequences commonly used in molecular biology, such as for example the “Myc” or “FLAG” tags mentioned below.

The term “ligand-receptor pair” denotes two bonding partners such as the pairs: haptene/antibody; DNP/anti-DNP antibody, in which the DNP represents dinitrophenol; GST/anti-GST antibody in which GST represents glutathione S-transferase; biotin/avidin; 6HIS/anti-6HIS antibody in which 6HIS is a peptide consisting of 6 histidines; Myc/anti-Myc antibody in which Myc is a peptide consisting of amino acids 410-419 of the human Myc protein; FLAG®/anti-FLAG® antibody in which FLAG® is a peptide having the 8 amino acids below: DYKDDDDK; HA/anti-HA antibody in which HA is an epitope of the influenza haemagglutinin, consisting of the 9 amino acids below: YPYDVPDYA. Other pairs may be used.

The DNA sequences of the pairs commonly called “tag/antitag” are well known to persons skilled in the art. These sequences are incorporated into plasmids or fused, by PCR, with the DNAs encoding the proteins of interest.

d) Measurement of the FRET Signal

The FRET signal may be measured in various ways: measurement of the fluorescence emitted by the donor alone, by the donor and the acceptor or measurement of the variation in the polarization of the light transmitted in the medium due to the FRET. It is also possible to incorporate the measurement of FRET by observing the variation in lifetime at the level of the donor, which is facilitated by the use of a donor with a long fluorescence lifetime, such as the rare-earth metal complexes (in particular on simple apparatus such as plate readers).

Preferably, the time-resolved FRET signal (TR-FRET signal) will be measured.

On the subject of the measurement of the FRET signal or of the TR-FRET signal, reference may be advantageously made to the documents incorporated below into the present description by way of references:
Mathis G. “Rare Earth Cryptates and Homogeneous Fluoroimmunoassays with Human Sera”, Clin. Chem. (1993) 39, No. 9, 1953-1959;
Mathis G. “Probing Molecular Interactions With Homogeneous Techniques Based on Rare Earth Cryptates and Fluorescence Energy Transfer”, Clin. Chem. (1995) 41, No. 9, 1391-1397;
H. Bazin et al., “Time resolved amplification of cryptate emission, a versatile technology to probe biomolecular interactions”, Reviews in Molecular Biotechnology (2002) 82, 233-250;

and to the patents or patent applications below: EP 321 353; EP 232 348; EP 539 477; EP 539 435; EP 569 496; EP 1 161 685; EP 1 166 119; WO 20044013348.

Finally, it will be noted that the selectivity of the measurement of energy transfer may be improved using the properties of polarization of the donor and acceptor fluorophores.

Thus, when the properties of polarization of the donor and acceptor fluorophores are used, the procedure is carried out as follows:

(i) the measurement medium is excited with a polarized light beam at the wavelength λ1, λ1 being the wavelength at which the said donor fluorophore is excited, and

(ii) the emitted fluorescence is measured at the wavelength λ3 in a plane of polarization different from the plane of polarization of the excitatory light, λ3 being the wavelength at which light is emitted from the acceptor fluorophore.

The measurement of the emitted signal at the wavelength of emission of the acceptor fluorophore in a different plane (that is to say non-parallel) from the plane of polarization of the excitatory light makes it possible to promote the measurement of the signal emitted by the highly depolarized species, and in particular the signal for the acceptor used in the transfer of energy. The plane in which the measurement is carried out is preferably the plane orthogonal to the plane of polarization of the excitatory light. Measurements in other planes may also be suitable.

According to a preferred aspect, the technique using the properties of polarization additionally comprises the following steps:

(iii) measurement of the emitted fluorescence at the wavelength λ2, λ2 being the wavelength at which light is emitted from the donor fluorophore, and

(iv) correction of the signal emitted by the acceptor fluorophore at the wavelength λ3 by the signal emitted by the donor fluorophore at the wavelength λ2.

This correction may for example consist in a calculation of the ratio of the intensity of the fluorescence measured at the wavelength λ3 to that measured at the wavelength λ2.

In the case where a measurement of fluorescence is carried out at the wavelength of emission of the donor (λ2), this measurement may be carried out in a plane that is parallel or different, preferably orthogonal to the plane of the excitatory light.

According to a second embodiment, the properties of polarization of the donor and acceptor fluorophores are used to improve the selectivity of the measurement of the transfer of energy, in a method aimed at determining the variation in polarization due to the transfer of energy. Like the first method described above, this method makes it possible to improve the selectivity of the measurement, which will thus be better correlated with the phenomenon of transfer of energy which it is desired to detect.

This second embodiment comprises the following steps:

(i) excitation of the measurement medium by a polarized light beam at the wavelength λ1, λ1 being the wavelength at which the said donor fluorophore is excited,

(ii) measurement of the total intensity of fluorescence (It_{//})_{λ2} emitted at the wavelength λ2 in the plane parallel to the plane of the excitatory light, λ2 being the wavelength at which light is emitted from the donor fluorophore,

(iii) measurement of the total fluorescence intensity (It_{⊥})_{λ2} emitted at the wavelength λ2 in a plane different from the plane of polarization of the excitatory light,

(iv) measurement of the total fluorescence intensity (It_{//})_{λ3} emitted at the wavelength λ3 in the plane parallel to the plane of excitatory light, λ3 being the wavelength at which light is emitted from the acceptor fluorophore,

(v) measurement of the total fluorescence intensity (It_{⊥})_{λ3} emitted at the wavelength λ3 in a plane different from the plane of polarization of the excitatory light,

(vi) calculation of the polarization P due to the transfer of energy between the donor fluorophore and the acceptor fluorophore according to the following formula:

$$P = \frac{[ {( {It}_{\text{//}} )}_{λ3} - {( {It}_{\text{//}} )}_{λ2} \times A \text{)} ] - G [ {( {It}_{⊥} )}_{λ3} - {( {It}_{⊥} )}_{λ2} \times B ) \text{]}}{[ {( {It}_{\text{//}} )}_{λ3} - {( {It}_{\text{//}} )}_{λ2} \times A \text{)} ] - nG [ {( {It}_{⊥} )}_{λ3} - {( {It}_{⊥} )}_{λ2} \times B ) \text{]}}$$

in which:

A represents the proportionality factor between the fluorescence emitted at the wavelengths λ2 and λ3 by the donor alone, in a plane parallel to the plane of the excitatory light,

B represents the proportionality factor between the fluorescence emitted at the wavelengths λ2 and λ3 by the donor alone, in a plane different to the plane of polarization of excitatory light,

n=1 or 2. When n=1, measurement of polarization is said to occur; when n=2, anistropy is involved.

G is a factor which makes it possible to correct the difference in sensitivity of detection in the parallel and orthogonal planes. This factor is either provided by the manufacturer, or can be easily determined by persons skilled in the art by measuring the polarization of substances of known polarization. In a particular embodiment, G is between 0.1 and 2, preferably G is between 0.8 and 1.2, and in particular G=1;

and

(vii) comparison of the calculated P value with that obtained in a control measurement medium in which transfer of energy does not occur, a decrease in P indicating an energy transfer.

According to a preferred embodiment, A and B are calculated in the following manner:
A=(Id_{//})_{λ1}(Id_{//})_{λ2}
B=(Id_{⊥})_{λ3}(Id_{⊥})_{λ2}
(Id_{//})_{λ3}, (Id_{//})_{λ2}, (Id_{⊥})_{λ3}, (Id⊥)_{λ2} corresponding to the intensities of fluorescence emitted at the wavelengths λ2 or λ3, in the planes that are parallel or different from the plane of polarization of the excitatory light, by a measurement medium containing the said donor fluorophore but not containing the acceptor fluorophore.

As in the first method described, the measurements carried out in a plane different from the plane of polarization of excitatory light are preferably carried out in the plane orthogonal to the plane of polarization of the excitatory light. Measurements in other planes could also be suitable, as long as the chosen plane is not the plane parallel to the plane of polarization of the excitatory light.

The method according to the invention therefore makes it possible to improve the selectivity of the measurement of a phenomenon of transfer of energy between a donor compound and an acceptor compound. This is particularly advantageous in the case where the spectral selectivity between the donor and the acceptor is not optimum, that is to say in the following cases:

cases where the emission spectra of the donor and the acceptor overlap. The methods according to the invention are particularly effective in the case where 5 nm<λ3−λ2<100 nm, λ3−λ2 representing the difference between the wavelengths λ3 and λ2.

cases where a direct parasitic excitation of the acceptor is possible at the wavelength of excitation of the donor (λ1).

In a preferred aspect, the donor and acceptor fluorophores have a high polarization, in particular greater than 50 mP, preferably greater than 100 mP. The donor and acceptor compounds whose intrinsic polarization is less than 50 mP may be coupled or adsorbed to carrier molecules (organic molecules, proteins, peptides, antibodies, or other molecules as described below), which will have the effect of increasing the apparent polarization of the fluorophore and will make it possible to use it in the methods according to the invention.

In another preferred aspect, the donor and acceptor fluorophores are chosen such that, following excitation at the excitation wavelength of the donor λ1, no emission of the acceptor is detected at the wavelength of emission of the donor λ2.

### BRIEF DESCRIPTION OF THE DRAWINGS

FIG. 1 illustrates mechanisms for activation of GPCRs in the presence of ligand, and

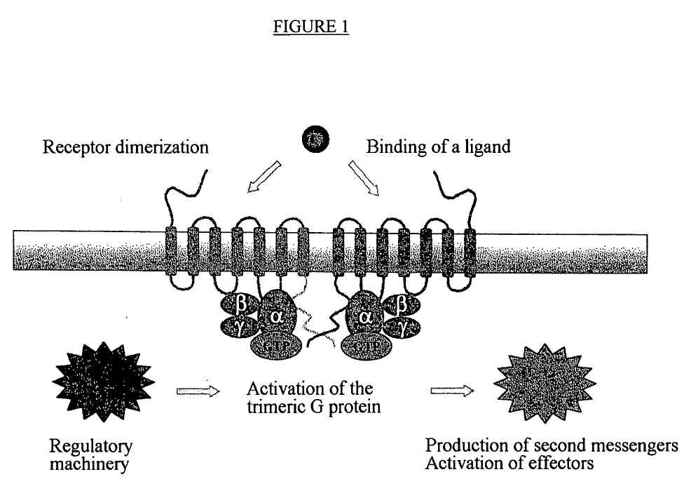

FIG. 2 shows results of proximity transfer as measured by FRET signal.

The invention will now be described in more detail by the illustrative but nonlimiting examples below.

### EXAMPLE 1

Tags were inserted by PCR in the C-terminal parts of the two subunits GB₁ and GB₂ (Myc tag: EQKLISEEDL), after the residue E916 for GB1 and after the residue E793 for GB2, forming the functional receptor GABAB, and between the residues V93 and E94 in the α subunit of the Go protein (FLAG tag: DYKDDDDK).

The various constructs are expressed in HEK293 cells transiently transfected by electroporation (BioRad electroporator) at the rate of 10 million cells per experimental conditions, electric shock at 260 volts for a capacitance of 1 mF. The cells are then subcultured in complete DMEM culture medium containing 10% fcetal calf serum. The cells are then distributed at the rate of 100 000 cells/well in a 96-well plate.

24 hours after electroporation, the cells are then incubated, after rinsing in PBS buffer, for at least 5 hours at 4° C. (in order to reach an equilibrium state) with a mixture of anti-Myc antibody (3 nM final) and anti-FLAG antibody (1 nM final), labelled with AlexaFluor647 (molecular probes) (acceptor) and labelled with europium cryptate (PyridineBispyridine PBP) (donor), respectively. The reaction medium containing the antibodies is the following: 5 mM HEPES, 11 mM EGTA, 2 mM MgCl₂, 10 mM NaCl, 120 mM KCl, 1 mM CaCl₂, pH 7.3, Triton X100 0.01%). This medium has an isoosmotic composition compared with the intracellular medium. The low Triton X100 concentration brings about controlled permeabilization of the cells, which allows the antibodies to reach the intracellular epitopes without having to fix the cells beforehand.

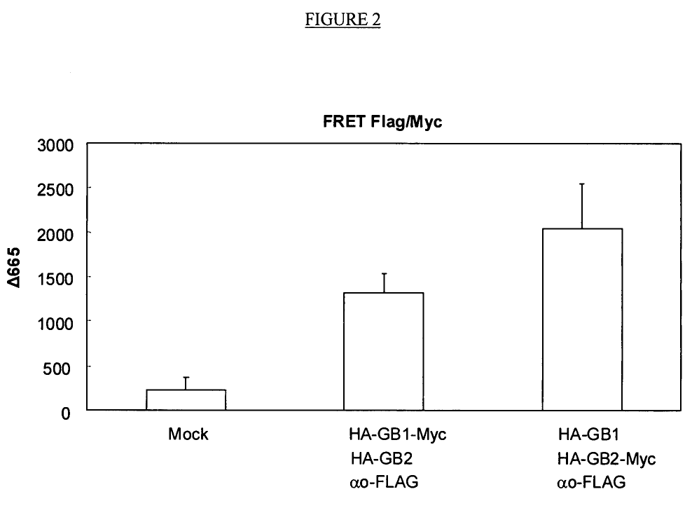

The results obtained are represented in the appended FIG. 2.

The measurements of FRET signal, represented by the Δ665 signal (emission at 665 nm of the acceptor corrected for the background noise), between the Myc tags at the C-terminals of GB₁ or GB₂ and the Go-FLAG protein, demonstrate a specific interaction between the two proteins. The FRET signal may be quantitatively measured on a fluorescence reader (Rubystar).

By way of comparison, the FRET signal was also measured for cells transfected with the same final quantity of plasmids as in the experimental points. Under this condition, called mock, the plasmid does not contain coding sequences for either of the GB1, GB2 or Go proteins.

### EXAMPLE 2

The coding sequences for the SNAPTag and HaloTag enzymes were respectively inserted, by molecular biology, in the C-terminal part of the subunits GB₁ or GB₂ (forming the functional receptor GABAB), and in the loops identified in the Gβγ subunits of the Go protein. These constructs were expressed in the HEK293 cells transiently transfected as indicated in Example 1. 24 hours after transfection, the cells are incubated in an incubator at 37° C., 5% CO₂, for 30 minutes with culture medium containing 5 μM of each substrate specific to the enzymes SNAPTag and HaloTag, labelled with an acceptor fluorophore (A) and labelled with a donor fluorophore (D), respectively. After washing, the cells are again incubated for 30 minutes in their culture medium in an incubator at 37° C., 5% CO₂ before carrying out the detection of the signal. The result of the SNAPTag and HaloTag enzymatic activities is a covalent incorporation of the donor and acceptor fluorophores into the functional GPCR of interest and into the Gβγ subunit of the Go protein, respectively. The measurements of signal may be carried out quantitatively with the aid of a fluorescence reader (RubyStar) or qualitatively and semiquantitatively with the aid of an epifluorescence microscope.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited herein and of corresponding U.S. Provisional Application Ser. No. 60/651,987, filed Feb. 14, 2005, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Method for the detection of the interactions between a G protein-coupled receptor (GPCR) and one of the Gα or Gβγ subunits of a G protein, which comprises the steps consisting in:
1) bringing a receptor labelled with a member of a donor/acceptor pair into contact with a Gα or Gβγ subunit of a G protein labelled with the other member of the donor/acceptor pair;
2) measuring the transfer by proximity effect between the donor and the acceptor.

2. Method according to claim 1, characterized in that the bringing into contact is carried out by transfecting cells with a nucleic acid encoding a receptor and with a nucleic acid encoding a Gα or Gβγ subunit of a G protein, the receptor and the Gα or Gβγ subunit being coupled to a member of a donor/acceptor pair, for one, and to the second member of the said donor/acceptor pair, for the other.

3. Method according to claim 1, characterized in that the bringing into contact is carried out either in the presence of a natural ligand for GPCR, or in the presence of a potential pharmacological agent or of a test molecule.

4. Method according to claim 1, characterized in that the measurement of the transfer by proximity effect is carried out as follows:
1) the transfer by a proximity effect between the donor and the acceptor is measured in the absence of ligand, of pharmacological agents for the said receptor and of test molecules (basal signal);
2) the signal obtained in the presence of a specific ligand for this GPCR which will trigger the activation of this receptor (reference signal) is optionally compared to the basal signal;
3) the signal obtained in the presence of pharmacological agents or of chemical molecules which are capable of modulating the activity of the said receptor is compared to the basal and reference signals.

5. Method according to claim 1, characterized in that the members of the donor/acceptor pair are fluorophores, in that the proximity transfer is a transfer of fluorescence energy and in that the measurement of the transfer of energy is carried out by measuring the fluorescent signal resulting from a transfer between the donor and the acceptor (FRET signal).

6. Method according to claim 5, characterized in that the donor fluorophore and the acceptor fluorophore are chosen from rhodamines, cyanines, squaraines, the fluorophores known by the name BODIPY, fluoresceins, the compounds known by the name AlexaFluor, rare-earth metal chelates, rare-earth metal cryptates, Quantum dots, fluorescent proteins such as the green fluorescent protein (GFP) or its variants, fluorescent proteins extracted from corals, phycobiliproteins, such as B-phycoerythrin, R-phycoerythrin, C-phycocyanin, allophycocyanins, in particular those known by the name XL665.

7. Method according to claim 5, characterized in that the donor fluorophore is a rare-earth metal complex.

8. Method according to claim 5, characterized in that the rare-earth metal complex is a terbium or europium complex.

9. Method according to claim 5, characterized in that the rare-earth metal complex is a chelate or a cryptate.

10. Method according to claim 9, characterized in that the rare-earth metal cryptate is a cryptate having a pyridine unit.

11. Method according to claim 10, characterized in that the cryptate has a pyridine unit.

12. Method according to claim 5, characterized in that the acceptor fluorophore is chosen from allophycocyanines, cyanines, rhodamines, squaraines, BODIPYs, fluoresceins, Alexas.

13. Method according to claim 5, characterized in that the acceptor fluorophore is a green fluorescent protein (GFP) or one of its variants, the yellow fluorescent protein (YFP) or the blue fluorescent protein (CFP), the fluorescent proteins extracted from corals.

14. Method according to claim 5, characterized in that the coupling between the GPCR receptor or the Gα or Gβγ subunit and the donor fluorophore or the acceptor fluorophore is a direct coupling by covalent bonding or an indirect coupling.

15. Method according to claim 14, characterized in that the coupling by a covalent bond is carried out by fusion with a protein sequence having an irreversible (“suicide”) enzymatic activity or by splicing with the aid of an intein.

16. Method according to claim 15, characterized in that the GPCR receptor, the Gα subunit or the Gβγ subunits contain, as a fusion, a peptide sequence called extracellular or intracellular tag for GPCR and intracellular tag for the Gα subunit or the Gβγ subunits, and in that the coupling by indirect bonding is carried out by means of antibodies specifically recognizing the said tags carried by the said GPCR receptor, the said Gα subunit or the said Gβγ subunits.

17. Preparation of cells stably or transiently transfected with a nucleic acid encoding a GPCR and with a nucleic acid encoding a Gα or Gβγ subunit of the corresponding G protein, the GPCR receptor and the Gα or Gβγ subunit being coupled to a donor fluorophore, for one, and to an acceptor fluorophore, for the other.

18. Preparation of transfected cells according to claim 17, characterized in that the donor fluorophore is a rare-earth metal complex.

19. Preparation of transfected cells according to claim 18, characterized in that the rare-earth metal complex is a terbium or europium complex.

20. Preparation of transfected cells according to claim 18, characterized in that the rare-earth metal complex is a chelate or a cryptate.

21. Preparation of transfected cells according to claim 20, characterized in that the rare-earth metal cryptate is a cryptate having a pyridine unit.

22. Preparation of transfected cells according to claim 17, characterized in that the acceptor fluorophore is chosen from allophycocyanines, cyanines, rhodamines, squaraines, BODIPYs, fluoresceins, Alexas.

23. Preparation of transfected cells according to claim 22, characterized in that the acceptor fluorophore is the yellow fluorescent protein (YFP) or the green fluorescent protein (GFP).

24. Preparation of transfected cells according to claim 23, characterized in that the coupling between the GPCR receptor or the Gα or Gβγ subunit and the donor fluorophore or the acceptor fluorophore is a direct coupling by covalent bonding.

25. Preparation of transfected cells according to claim 24, characterized in that the coupling by a covalent bond is carried out by fusion.

26. Preparation of transfected cells according to claim 25, characterized in that the GPCR receptor contains an extracellular or intracellular tag and in that the coupling by a covalent bond is carried out by means of the antibody recognizing the extracellular or intracellular tag of the said receptor.
